(19) United States
(12) Patent Application Publication
(10) Pub. No.: US 2026/0317791 A1
(43) Pub. Date: Oct. 15, 2026

(54) METHOD OF REDUCING EMULSION FORMATION DURING FERMENTATION PROCESS

(71) Applicant: DSM IP Assets B.V., Maastricht (NL)

(72) Inventor: Negash ADUGNA, Kaiseraugst (CH)

(21) Appl. No.: 19/482,658

(22) PCT Filed: May 14, 2024
(86) PCT No.: PCT/EP2024/063229

§ 371 Date: Nov. 7, 2025

Related U.S. Application Data
(60) Provisional application No. 63/466,362, filed on May 15, 2023.

(30) Foreign Application Priority Data
May 31, 2023 (EP) 23176436.6

(51) Int. Cl. C12P 5/00

(52) U.S. Cl. CPC C12P 5/007

(57) ABSTRACT

A method for the production of farnesene oil comprising fermenting yeast with feed stock in a fermentation vessel to produce a whole cell broth (WCB); and adding a surfactant to the fermentation vessel to reduce emulsification inside fermenter.

### BACKGROUND

### 1. Field

The present disclosure relates to methods for improved recovery of terpinoids, lipophilic isoprenoids, lipids, and biomass from fermentation broth, by reducing emulsion formation, comprising blocking emulsion formation and/or simultaneously demulsifying emulsion in a fermentation vessel during a fermentation process, the method further comprising adding a surfactant to the fermentation vessel during the fermentation process. The method also affects the surface chemistry of the cell and its interaction with fermentation media, whereby the separation of the biomass is simplified.

### 2. Description of Related Art

Terpenoids, lipids, and biomass can be produced by microorganisms like yeast and microalgae via fermentation, where the cells make oil and biomass containing intracellular or extracellular products that turn into emulsion inside the fermenter. To recover the target product, the fermented broth is centrifuged and separated into a Heavy Phase (HP) and a Light Phase (LP). The HP mainly contains cell biomass and part of fermentation aqueous medium. The LP contains product in the form of oil, emulsion and part of fermentation medium. In a particular process, the recovery of the product from the light phase is done by adding surfactant under mixing and heating in order to break the emulsion. The combination of the Light Phase (LP) and the surfactant is sent to a centrifuge or other separation mechanism to recover product.

A problem in the fermentative production of organic compounds is that it is difficult to recover the organic compounds from the fermentation mixture. Separation of the organic compound from the fermentation mixture often relies on the use of a surfactant to break emulsions comprising the organic compound. However, adding a surfactant adds cost and the surfactant becomes an impurity that must be removed via downstream processes (DSP). Moreover, some emulsions are not broken by addition of a surfactant and some emulsions are only partly broken by addition of a surfactant. Further steps or additional conditions required to break an emulsion, such as addition of heat, carry the risk of creating further impurities. There is a need for recovering fermentation products from a culture medium in high yields using reduced amounts of surfactants.

### BRIEF SUMMARY

In an embodiment, a method for reducing emulsion formation inside a fermenter during the production of organic compounds can comprise fermenting a microorganism with a feed stock in a fermentation vessel to produce a whole cell broth (WCB) comprising organic compounds; adding a surfactant to the feed stock, whole cell broth, to a portion of the whole cell broth, or a combination thereof, in the fermentation vessel; optionally, removing a portion of the whole cell broth; and heating the fermentation vessel.

In an embodiment, the microorganism is a bacteria. The bacteria can be an Escherichia, optionally E. coli, Bacillus or Lactobacillus species.

In an embodiment, the microorganism is a fungi, optionally a yeast. The yeast can be a Saccharomyce, optionally S. cerevisiae. or Pichia species.

In an embodiment, the microorganism is an algae, optionally as Chlorella species.

In an embodiment, the microorganism is a recombinant microorganism.

In an embodiment, the surfactant is selected from an anionic surfactant, a non-ionic surfactant, or a combination thereof.

In an embodiment, the surfactant is an anionic surfactants selected from sulfates and sulfonates, sodium dodecylsulfate (SDS), sodium dodecylbenzene sulfonate, a disulfonate, sodium dodecylnaphthalene sulfate, dialkyl benzenealkyl sulfates and sulfonates, acids optionally abitic acid, alkyldiphenyloxide disulfonate including two C6-C16 hydrocarbon chains branched on each of the sulfonated aromatic rings, and combinations thereof.

In an embodiment, the surfactant is a non-ionic surfactant selected from polyether polyols, optionally copolymers of propylene oxide (PO) and ethylene oxide (EO), including those made by capping polypropylene glycols with ethylene oxide. The surfactant can be a non-ionic surfactant is a polyether glycol having a molecular weight of at least 2400 g/mol, less than 2800 g/mol, or less than 3000 g/mol. The anionic surfactant can be selected from sulfates and sulfonates.

In an embodiment, the nonionic surfactant is a polyether polyol. The surfactant can be L-62 (polyether polyol). The surfactant can be L-81 (polyether polyol).

In an embodiment, the surfactant is added in an amount of between about 0.001% v/v (volume/volume) and 1% v/v (volume/volume). The surfactant can be added in an amount of between about 0.01 and 1% v/v. The surfactant can be added in an amount of about 0.01, 0.02, 0.03, 0.04, 0.05, 0.06, 0.07, 0.08, 0.09, or 1% v/v. The surfactant can be added in an amount of between about 0.02 and 0.06% v/v, 0.03 and 0.05% v/v or 0.03 and 0.04% v/V.

In an embodiment, the surfactant is added in the form of a solid, optionally in crystalline, amorphous, pelletized, granulated form, or a combination thereof. The surfactant can be added in the form of a pure form, a solution, optionally, a dilute solution, a saturated solution, or a super-saturated solution.

In an embodiment, the fermentation is for between about 1 and 14 days. The fermentation is for about 1, 2, 3, 4, 5, 6, 7, 8, 9, 10, 11, 12,13, or 14 days.

In an embodiment, the portion of the whole cell broth is removed after 1 day. The portion of the whole cell broth can be removed after about 1, 2, 3, 4, 5, 6, 7, 8, 9, 10, 11, 12,13, or 14 days.

In an embodiment, step (b) is for about 24 hours.

In an embodiment, the method further comprises isolating organic compounds from the whole cell broth.

In an embodiment, the organic compounds comprise isoprenoids (terpenoids).

In an embodiment, the method further comprises isolating an isoprenoid.

In an embodiment, the isoprenoid is (a) selected from the group consisting of a hemiterpene, monoterpene, sesquiterpene, diterpene, triterpene, tetraterpene, and polyterpene, or (b) an isoprenoid which is not a carotenoid, or (c) a C₅-C₂₀ isoprenoid, or (d) selected from the group consisting of abietadiene, amorphadiene, carene, α-farnesene, β-farnesene, farnesol, geraniol, geranylgeraniol, isoprene, linalool, limonene, myrcene, nerolidol, ocimene, patchoulol, β-pinene, sabinene, γ-terpinene, terpinolene and valencene. The isoprenoid can be a C₅-C₂₀ isoprenoid. The isoprenoid can be a hemiterpene, optionally isoprene. The isoprenoid can be a monoterpene. The monoterpene can be selected from carene, geraniol, linalool, limonene, myrcene, ocimene, β-pinene, sabinene, γ-terpinene, terpinolene, or a combination thereof. The isoprenoid can be a sesquiterpene isoprenoid. The sesquiterpene isoprenoid can be selected from abietadiene, amorphadiene, farnesene, optionally α-farnesene, β-farnesene, or a combination thereof, farnesol, nerolidol, patchoulol, valencene, and combinations thereof. The isoprenoid can be farnesene. The isoprenoid can be α-farnesene, β-farnesene or a mixture thereof. The isoprenoid can be a diterpene, optionally geranylgeraniol.

In an embodiment, the method can further comprise separating the whole cell broth into an organic phase comprising the isoprenoid compound and a heavy phase comprising solid and liquid fermentation medium.

In an embodiment, the method can further comprise separating the whole cell broth into a liquid phase comprising the isoprenoid compound and a solid phase comprising microorganisms, cell debris.

In an embodiment, the method can further comprise separating the liquid phase comprising the isoprenoid compound into a light phase comprising the isoprenoid compound and heavy phase comprising dead cell layer, cell debris, and fermentation aqueous phase.

In an embodiment, the method can further comprise further purification of an isoprenoid.

In an embodiment, the method comprises an air feed to fermenter between about from 3 L/minutes to 10 L/minutes.

In an embodiment, the dissolved oxygen in the fermenter can be maintained at above about 15%.

In an embodiment, the dissolved oxygen in the fermenter can be maintained at 0%.

In an embodiment, the surfactant reduces emulsion formation. The surfactant can reduce emulsion formation between about 1% and 100% as compared to emulsion formation without the addition of the surfactant. The surfactant can reduce emulsion formation between about 10%, 20%, 30%, 40%, 50%, 60%, 80%, or 90% as compared to emulsion formation in a fermenter without the addition of the surfactant.

In an embodiment, the surfactant reduces the amount of dead cells (dead cell layer) in the whole cell broth. The surfactant can reduce the amount of dead cells (dead cell layer) by between about 1% and 50%.

In an embodiment, the surfactant blocks emulsion formation.

### BRIEF DESCRIPTION OF THE DRAWINGS

The patent or application file contains at least one drawing executed in color. Copies of this patent or patent application with color drawing(s) will be provided by the Office upon request and payment of the necessary fee.

FIG. 1 depicts 14 mL samples from 10 L fermenters at the 8^{th} withdrawal and centrifuged at 11,000×g for 3 minutes (1^{st} picture=sample from control fermenter that was sparged at 3 l/min; 2^{nd} picture sample from second control fermenter but air sparged at 6 l/min; 3^{rd} picture=sample from one time L62 addition inside fermenter (one time addition between each withdrawals); 4^{th} picture=sample from L62 addition inside fermenter (L62 addition every 2 hours till harvest) all samples centrifuged at 11,000×g for 3 minutes. FIG. 2-1: sample centrifuged from fermenter NB5 where no surfactant added-(control); FIG. 2-2: sample centrifuged from fermenter NB6 where 25% withdrawal was taken out instead of the regular 30-40% withdrawal for the other fermenters. FIG. 2-3: sample centrifuged from fermenter NB7 where one time L-62 addition b/n withdrawals; and FIG. 2-4: sample centrifuged from fermenter NB8 where semi-continuous L-62 addition b/n withdrawals.

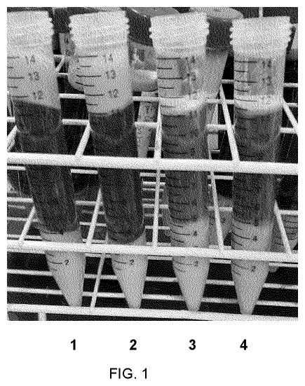

FIG. 2 depicts % v/v of biomass for samples from fermenter NB5 (control), from fermenter NB7 (one-time addition of L62 between withdrawals) and from fermenter NB8 (semi-continuous addition of L62). The FIG. also depicts the % v/v oil from one-time L62 addition (NB7) and % v/v oil from semi-continuous addition of L62 (NB8). The FIG. also depicts surfactant added in grams inside the one-time L62 addition between withdrawals for NB7 and a semi-continuous addition of L62 inside fermenter NB8.

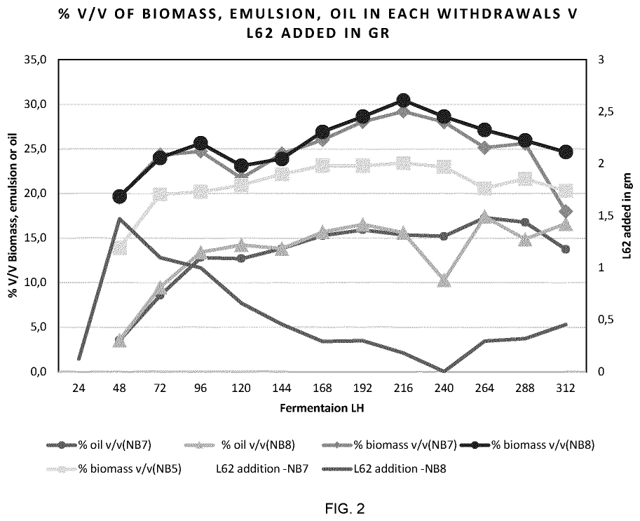

FIG. 3 depicts estimated amount of emulsion in each withdrawals in the control fermenter NB5, estimated amount of crude oil in each withdrawals in fermenter NB7 (one-time addition of L62 between withdrawals) and estimated amount of crude oil in each withdrawals in fermenter NB8 (semi-continuous addition of L62). This Figure also depicts surfactant added in grams inside fermenter NB7 (the one-time L62 addition between withdrawals) and surfactant added in grams inside fermenter NB8 (semi-continuous addition of L62 inside fermenter.

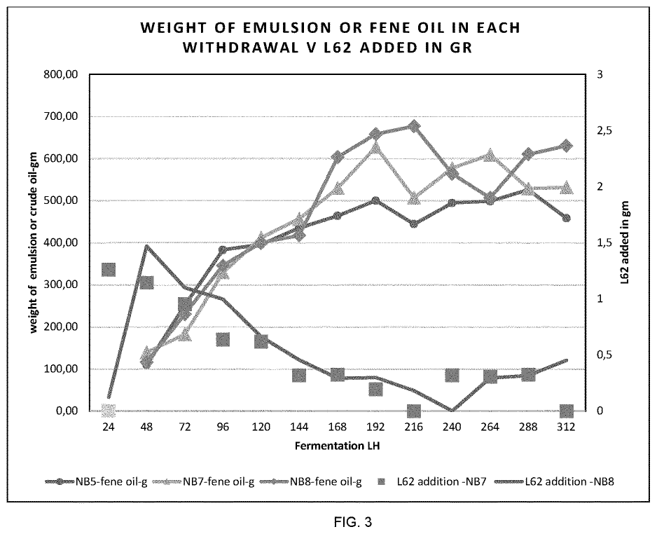

FIG. 4 depicts the four main phases after demulsification and centrifugation. The top layer shows Farnesene containing crude oil. The 2^{nd} layer from top shows the dead cell Layer (DCL). The 3^{rd} from top shows the aqueous phase mainly containing aqueous media from fermentation. The 4^{th} layer from top is mainly the biomass and some solids from fermentation process.

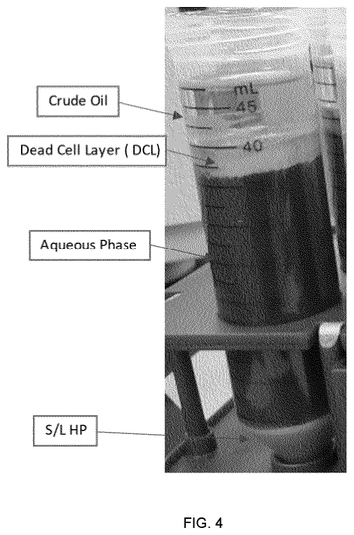

### DETAILED DESCRIPTION

Before the subject disclosure is further described, it is to be understood that the disclosure is not limited to the particular embodiments of the disclosure described below, as variations of the particular embodiments can be made and still fall within the scope of the appended claims. It is also to be understood that the terminology employed is for the purpose of describing particular embodiments and is not intended to be limiting. Instead, the scope of the present disclosure will be established by the appended claims.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this disclosure belongs.

### Definitions

“Organic compound,” “bio-organic compound,” or “microbial-derived organic compound” as used herein refers broadly to an organic compound that is made by microbial cells, including recombinant microbial cells as well as naturally occurring microbial cells.

“Cell,” as used herein refers broadly to a microorganism, capable of being grown in a liquid growth medium.

“Dry weight” or “dry matter” as used herein refers broadly to weight determined in the relative absence of water. For example, reference to cells as comprising a specified percentage of a particular component by dry weight means that the percentage is calculated based on the weight of the cell after substantially all water has been removed (until constant weight).

“Dry cell weight” or “dry cell matter” or “total suspended solids” as used herein refers broadly to weight determined in the relative absence of water after sample washing for insoluble solids removal.

“Isoprenoid compound” as used herein refers broadly to compounds capable of being derived from isopentenyl diphosphate (IPP).

“Microbial cell” refers to organisms such as algae, bacteria, fungi, protest and combinations thereof, e.g., unicellular organisms.

“Cell-associated” as used herein in connection to fermentation products refers broadly to fermentation products that are associated to the host cell or host cell debris.

“Emulsion” as used herein refers broadly to a mixture of two immiscible liquids, such as water and an oil. As used herein, it particularly refers to a mixture of a organic compound envisaged herein and water.

“Host cell” as used herein refers broadly to a microbial cell which is used for the production of a organic compound. The host cell can be a recombinant cell, which implies that it has been genetically modified to induce or increase the production of the organic compound. The host cell contains a foreign DNA and/or has one or more genetic modifications compared to the wildtype organism which affects the production of the organic compound. However, also considered host cells are microbial cells naturally producing an organic compound of interest.

“Surfactant” as used herein refers broadly to a compound that reduces the surface tension (or interfacial tension) between two liquids or between a liquid and a solid.

### Farnesene Production Process

The current farnesene process comprises fermentation of genetically modified yeast microorganism to produce farnesene oil that forms emulsion in the fermentation broth. The emulsion in the fermenter is partially or wholly harvested in the form of whole Cell Broth (WCB) and down-stream processing (DSP) separates the biomass from the emulsion in the first stage solid/liquid (S/L) centrifugation step and carry forward Clarified Cell Broth (CCB) that contains emulsion, liquid fermentation broth, proteinaceous material, dead cell and cell debris. In the next step, the CCB is heated to 75-80° C. and L-62 surfactant is added inline and mixed in a tank. This surfactant and CCB mixture is then centrifuged, and crude oil separated. The crude oil comprises organic compounds, for example isoprenoids (e.g., Farnesene oil). As the process is continuous, the emulsion composition differs from withdrawal to withdrawal and it is difficult to add consistently, proportional amounts of surfactant. This inconsistency in the demulsification process and oil in the dead cells results in about 8-10% product losses observed in the liquid/liquid (L/L) Heavy phase.

The crude oil obtained also contains surfactant residue and it is required to distill the crude oil to separate the farnesene oil from the surfactant. This added additional process cost. The evaporator also became a bottle neck to increase capacity. The inventors sought to block emulsion formation in the fermenter or complete demulsification inside the fermenter by adding surfactant inside the fermenter during fermentation process.

### Reduced Emulsion Formation

In the methods described herein, the goal is to eliminate or reduce emulsion formation at the source and/or demulsify any emulsion as it forms by adding surfactant to the fermentation vessel during the fermentation process. The addition of the surfactant can be a onetime addition between withdrawals, at harvest, or can be addition of specified amounts of surfactant over the duration of the fermentation process (e.g., regular, periodic addition of discrete amounts of surfactant). The methods described herein block emulsion formation and/or accomplish demulsification inside the fermentation vessel. The methods described herein reduce the consumption of the surfactant needed to produce farnesene oil by up to 91% as compared to current process. The methods described herein provide improved downstream biomass recovery. Additionally, the inventors found unexpectedly that fermentation productivity of Farnesene oil was more than doubled when a sufficient amount of dissolved oxygen was supplied. The inventor also found the amount of dead cell in the withdrawals or harvested whole cell broth reduced which indicates the surfactant reduced the shearing effect on the cell during fermentation.

The methods described herein facilitate the separation and recovery of terpenoid, lipid oils, biomass and any other products that make emulsions or similar compositions during fermentation processes. The recovered terpenoids, and other oils, can be used as precursors for making other products, including vitamins, pharmaceuticals, perfumery, cosmetics, animal feed, and fuel oils. The methods described herein can also be used to concentrate and recover microbial organisms for intracellular product separation, purification or where the microbial organisms are used as product, like animal feed. The methods described herein reduce chemical consumption, waste reduction, and cost, and improve the quality of the products.

The methods described herein solve the problems related to the demulsification process or biomass separation process by blocking emulsion formation or by demulsifying simultaneously, as emulsion forms. The methods described herein reduce the problem of long fermentation processes by increasing productivity, solve the problem of long hours of biomass and emulsion separation, mixing, heating, and multiple stage centrifugation, and reduce the costs of material transport. The methods described herein significantly reduce chemical consumption associated with demulsification of fermentation products. The methods described herein eliminate or reduce the processes needed to separate surfactant from the final product. The methods described herein also reduce the environmental footprint by eliminating the need for evaporation columns, reducing the need for wastewater treatment, and reducing or eliminating other environmental issues related to chemicals and salts currently used in state-of-the-art methods.

An advantage of the methods described herein is to eliminate or minimize emulsion formation and to demulsify in the fermenter if an emulsion forms. This eliminates the need for heating and chemical addition to the fermentation broth for demulsifying in downstream steps and hence reduces the chemical consumption significantly. Another advantage of the methods described herein is to decrease surfactant consumption by up to 91%. Other advantages of the methods described herein include higher quality products and elimination of additional separation and purification steps like evaporation or distillation. The methods described herein reduce environmental footprints by reducing disposal of chemicals into the environment. The methods described herein reduce product waste in the waste stream and improve productivity and recovery.

### L-62 Addition Inside Fermenter During Fermentation

The methods described herein comprise the addition of a surfactant (L-62) during fermentation, which reduces emulsion formation, including the complete demulsification in the fermenter. Using the method described herein, a reduced surfactant consumption of up to 91% is achieved over comparative methods that add surfactant at downstream processing to demulsify the emulsion.

The addition of L-62 during fermentation showed 2-4% better overall recovery than current processes and 6-8% better recovery than if adding L-62 to the Harvest Tank. The outcome of L-62 addition in the fermenter reduces two-step centrifugations to a single step centrifugation. The L/L centrifuge can be used as a purification step, based on the crude oil characteristics from solid/liquid (S/L) centrifuge. The success of adding L-62 into the fermenter eliminates the need for heating CCB, for example to 75-80° C. The product loss into the L/L Heavy Phase stream dead cell Layer (DCL) separated at L/L centrifuge reduced to ~3-6%, from 8-10% loss for current process. The addition of surfactant inside fermenter reduced cell death by reducing the shearing effect on the biomass.

### Farnese Oil Production

The methods described herein provide a process for recovering an isoprenoid compound produced by fermentation from a fermentation medium, comprising: (a) fermenting a microorganism that produces isoprenoids; (b) (after a period of fermentation) demulsifying the fermentation medium or a fraction thereof in presence of a surfactant in the fermenter, thereby generating a stream having an organic phase containing the isoprenoid compound and a phase heavier than the organic phase, and (c) separating the stream obtained in step (b) into the organic phase (“light phase”) containing the isoprenoid compound, the phase heavier than the organic phase, and, optionally, a solid phase (“heavy phase”) containing host cells and cell debris.

The pH range of the fermentation medium or the fraction thereof is from 4 to 7. The pH range of the fermentation medium or the fraction thereof is from 4.8-5.2 and preferably 5.0.

The surfactant is selected from an anionic surfactant, a non-ionic surfactant or a combination thereof. The anionic surfactant is selected from sulfates and sulfonates and the non-ionic surfactant is a polyether polyol. Both are preferably soluble in water at room temperature (20° C.). Preferably, the surfactant is L-62, also called TERGITOL™, and is ≥99.0% polyalkylene glycol (CAS 9003-11-6).

The concentration of surfactant in the fermentation medium or the fraction thereof in the emulsion reducing step (a) is from about 0.01 to 1.0% v/v. For example, the concentration of surfactant in the fermentation medium or the fraction thereof in the emulsion reducing step (a) can be about 0.01, 0.02, 0.03, 0.04, 0.05, 0.06, 0.07, 0.08, 0.09, or 1.0% v/v. The concentration can be about 0.03, 0.04, or 0.05% v/v. The concentration can be between about 0.01 and 0.05% v/v or 0.02 and 0.06% v/v.

The separation step (b) can be a solid/liquid separation followed by liquid/liquid purification step.

The methods described herein can also comprise the recovery of isoprenoid as a fermentation product from the fermentation medium comprising:
(a) adding a surfactant to the fermentation vessel and fermenting an isoprenoid producing microorganism in a vessel;
(b) heating the fermentation vessel to between about 25° C. and 35° C.;
(c) harvesting all or a portion of the fermentation broth;
(d) separating, optionally by centrifugation, to produce a heavy phase containing solid comprising microorganism, cell debris and cell material and liquid fermentation medium and a light phase containing mainly organic phase containing mainly isoprenoid compound (and optionally further purifying the crude oil containing mainly isoprenoid compound by distillation into a pure isoprenoid compound and small portion of impurities);
(e) further purifying the light phase, optionally by centrifugation, into the liquid organic phase comprising mainly isoprenoid and a small proportion of heavy phase containing solids and liquid fermentation medium; and
(f) optionally further purifying the crude oil comprising mostly isoprenoids by distillation into a pure isoprenoid compound and small portion of impurities.

### Microbial Fermentation

Isoprenoids can be produced by microbial fermentation. For example, farnesene, a sesquiterpene isoprenoid, can be produced by microbial fermentation. Typically, microorganisms are cultured under conditions suitable for the production of the organic compounds by the microbial host cells. Suitable conditions include many parameters, such as temperature ranges, levels of aeration and/or oxygenation, pH, and media composition. Each of these conditions, individually and in combination, is typically varied and/or optimized to allow the microbial host cell to grow and/or to ensure optimal production of the organic compound of interest. Exemplary culture media include broths or gels. The host cells can be grown in a culture medium comprising a carbon source to be used for growth of the host cell. Exemplary carbon sources include carbohydrates, such as glucose, fructose, cellulose, or the like, that can be directly metabolized by the host cell. In addition, enzymes can be added to the culture medium to facilitate the mobilization (e.g., the depolymerization of starch or cellulose to fermentable sugars) and subsequent metabolism of the carbon source. A culture medium may optionally contain further nutrients as required by the particular microbial strain, including inorganic nitrogen sources such as ammonia or ammonium salts, and the like, and minerals and the like. Other growth conditions, such as temperature, cell density, and the like are generally selected to provide an economical process. Temperatures during each of the growth phase and the production phase may range from above the freezing temperature of the medium to about 50° C. The fermentation can be conducted aerobically, anaerobically, or substantially anaerobically. Briefly, anaerobic conditions refer to an environment devoid of oxygen. Substantially anaerobic conditions include, for example, a culture, batch fermentation or continuous fermentation such that the dissolved oxygen concentration in the medium remains between 0 and 10% of saturation. Substantially anaerobic conditions also includes growing or resting cells in liquid medium or on solid agar inside a sealed chamber maintained with an atmosphere of less than 1 oxygen. The percent of oxygen can be maintained by, for example, sparging the culture with an N₂/CO₂ mixture or other suitable non-oxygen gas or gasses. The fermentation can be conducted continuously, batch-wise, or some combination thereof. Conventional fermentation bioreactors, shake flasks, test tubes, microtiter dishes, and petri plates can be used.

In a preferred embodiment, the invention therefore provides a method for the production of organic compounds, comprising
(a) fermenting a microorganism with a feed stock in a fermentation vessel to produce a whole cell broth (WCB) comprising organic compounds;
(b) adding a surfactant to the feed stock, whole cell broth, to a portion of the whole cell broth, or a combination thereof, in the fermentation vessel; and
(c) optionally, removing a portion of the whole cell broth;
(d) heating the fermentation vessel.
wherein the organic compounds comprise isoprenoids, and
wherein the method comprises an air or oxygen feed to fermenter.

As illustrated by the examples, preferably the air or oxygen feed to the fermenter comprises between about from 3 L/minutes to 10 L/minutes. Preferably the air or oxygen is supplied simultaneously with or after the addition of the surfactant. Preferably the air or oxygen feed is continuously applied. More preferably the air or oxygen feed is continuously applied during a period comprising at least 50% of the time, more preferably at least 70% of the time, still more preferably at least 90% of the time, even more preferably at least 95% of the time and most preferably during 100% of the time, during the period starting with the addition of the surfactant until the end of the fermentation. The air or oxygen feed is preferably supplied via a feed line separate from the feed line for the surfactant. Hence, the air or oxygen feed is preferably supplied in parallel to the surfactant feed. Preferably the amount of the air or oxygen feed increases over time. The air or oxygen feed may increase gradually or step-wise. Most preferably the air or oxygen feed starts simultaneously with or after the addition of the surfactant and subsequently increases over time. More preferably the amount of oxygen added increases over time. Preferably the method comprises an air feed to the fermenter followed by a pure oxygen feed to the fermenter. More preferably the method may comprise an air feed to the fermenter, preferably comprising between about from 3 L/minutes to 10 L/minutes, followed by a pure oxygen feed to the fermenter.

By pure oxygen is herein understood a gas comprising equal to or more than 99 vol. % oxygen, more preferably equal to or more than 99.6 vol. % oxygen.

Preferences for the microorganism, the surfactant and the isoprenoids are as described herein above and herein below. More preferably the surfactant is a polyether polyol, still more preferably a polyether polyol comprising propylene oxide (PO) and/or ethylene oxide (EO), most preferably a copolymer of propylene oxide (PO) and ethylene oxide (EO). Most preferably the isoprenoid is Farnesene.

In a preferred embodiment, the invention also provides a method for the production of organic compounds, comprising
(a) fermenting a microorganism with a feed stock in a fermentation vessel to produce a whole cell broth (WCB) comprising organic compounds;
(b) adding a surfactant to the feed stock, whole cell broth, to a portion of the whole cell broth, or a combination thereof, in the fermentation vessel; and
(c) optionally, removing a portion of the whole cell broth;
(d) heating the fermentation vessel.
wherein the organic compounds comprise isoprenoids, and
wherein dissolved oxygen in the fermenter is maintained at above about 15%.

The aeration of the medium can suitably be carried out by an air or oxygen feed as described above.

More preferably the method comprises an air or oxygen feed to fermenter whereafter the dissolved oxygen in the fermenter is maintained at above about 15%.

Preferences for the microorganism, the surfactant and the isoprenoids are as described herein above and herein below. More preferably the surfactant is a polyether polyol, still more preferably a polyether polyol comprising propylene oxide (PO) and/or ethylene oxide (EO), most preferably a copolymer of propylene oxide (PO) and ethylene oxide (EO). Most preferably the isoprenoid is Farnesene.

As illustrated by the examples, the aeration of the fermentation medium advantageously allowed for increased biomass and oil formation. As a consequence, the above methods according to the invention can preferably be methods comprising no demulsification step.

### Microbial Cells

Suitable microorganisms for fermentation are known in the art. Suitable microorganisms include but are not limited to bacteria, e.g., Escherichia (e.g., E. coli), Bacillus or Lactobacillus species, fungi, in particular yeasts such as Saccharomyces (e.g., S. cerevisiae) or Pichia species, or algae such as Chlorella species. The microbial host cell can be a fungus, preferably a yeast. The microorganisms can naturally produce the organic compound of interest, or they may have been genetically modified (e.g., recombinant microorganisms) to ensure production of the organic compound of interest, farnesene for example.

Suitable microorganisms for use in the methods described herein are capable to produce farnesene; in particular, the microorganism is capable excreting farnesene. Non-limiting examples of microorganisms suitable for the present invention are genetically modified host cells as described in WO 2013/071172, WO2014/144135, WO2008/039499, WO2007/140339, and WO2006/014837.

### Surfactant Addition

The methods described herein provide for reducing emulsion formation during fermentation (including complete demulsification inside the fermenter) and eliminates the need of demulsification step at downstream process. The methods can be performed by adding surfactant at start of the fermentation process, at the start of inoculation step or adding surfactant at log hour of 24. The methods can also be performed by adding surfactant one time between partial withdrawal of fermentation medium or adding surfactant in time intervals, for example in 1 hour interval, 2 hour interval, 3-5 hour interval, 4-10 hour interval, or 24 hour interval or 48 hour interval.

### Harvesting and Recovery of Isoprenoid Compound

The methods can also be performed by withdrawing fraction of the fermentation medium or harvesting whole of the fermentation medium. The harvested fermentation broth can be separated in Solid/Liquid centrifuges into fermentation medium fractions. The fermentation medium fraction can be a liquid stream obtained by extracting isoprenoid compounds from a solid/liquid heavy phase obtained from a solid/liquid separation of the fermentation medium into a solid/liquid heavy phase and a solid/liquid light phase. Some organic compounds are indeed associated to the cells and/or host cell debris contained in this solid/liquid heavy phase (e.g. located in an inner cell compartment or adsorbed on the cell wall).

The fermentation medium fraction can be a liquid stream obtained by extracting isoprenoid compounds from a liquid/liquid heavy phase. This liquid/liquid heavy phase is obtained from a liquid/liquid separation of a solid/liquid light phase into a liquid/liquid heavy phase and a liquid/liquid light phase. The solid/liquid light phase is the one obtained as already explained from the optional solid/liquid separation of the fermentation medium into a solid/liquid heavy phase and a solid/liquid light phase. This liquid/liquid heavy phase can be submitted to a second solid/liquid separation to be separated into a second solid/liquid light phase and a second solid/liquid heavy phase. Isoprenoid compounds are then extracted from the second solid/liquid light phase.

The fermentation medium fraction can be a liquid stream obtained by extracting isoprenoid compounds from the phase heavier than the organic phase or from the solid phase containing host cell debris and cells.

The fermentation medium or fraction thereof can comprise up to 35% v/v of cells (living or dead cells) and/or host cell debris. The % v/v of cells and/or debris can be measured by usual methods such as determination of volume ratios obtained by centrifugation in a capillary tube. For example, a fermentation medium may contain up to 35% v/v or up to 30% v/v of cells. In general, about 1-5% v/v are cells still containing the isoprenoid compound.

### Surfactants

Surfactants are usually organic compounds that are amphiphilic, which means they contain both hydrophobic groups and hydrophilic groups. Anionic surfactants contain anionic functional groups, such as sulfate, sulfonate, phosphate, and carboxylates. Suitable surfactants include, but are not limited to, non-ionic surfactants, anionic surfactants and combination thereof, in particular anionic surfactants which are water soluble.

Anionic surfactants include but are not limited to sulfates and sulfonates, sodium dodecylsulfate (SDS), sodium dodecylbenzene sulfonate, sodium dodecylnaphthalene sulfate, dialkyl benzenealkyl sulfates and sulfonates, acids such as abitic acid, combinations thereof. Other suitable anionic surfactants include, alkyldiphenyloxide disulfonate including two C6-C16 hydrocarbonchains branched on each of the sulfonated aromatic rings, such as, by example, DOWFAX™ 2A1 from Dow Chemical Company.

The surfactant is a sulfonate, in particular a disulfonate. Advantageously, this surfactant is soluble in water.

Examples of non-ionic surfactants include, for example, polyether polyols, in particular copolymers of propylene oxide (PO) and ethylene oxide (EO), including those made by capping polypropylene glycols with ethylene oxide, as those available from The Dow Chemical Company as TERGITOL™ L.

The non-ionic surfactant is a polyether glycol having a molecular weight of at least 2400 g/mol. In Particular, the molecular weight is less than 3000 g/mol, preferably less than 2800 g/mol. The molecular weight may have a value within a range defined by any combination of these limits.

The non-ionic surfactant is a polyether glycol having a molecular weight of at least 2400 g/mol. Advantageously, this surfactant is soluble in water.

Combinations of these surfactants and any of the foregoing surfactants can be in the methods described herein.

The surfactant can be added in an amount of between about 0.01 and 1% v/v. For example, the surfactant can be added in an amount of about 0.01, 0.02, 0.03, 0.04, 0.05, 0.06, 0.07, 0.08, 0.09, or 1% v/v. The surfactant can be added to be in an amount of between about 0.02 and 0.06% v/v, 0.03 and 0.05% v.v, or 0.03 and 0.04% v.v.

A surfactant can be introduced as a solid (e.g., in crystalline, amorphous, pelletized, and/or granulated form), and/or as a solution (e.g., a dilute solution, a saturated solution, or a super-saturated solution) containing, for example, water, an alcohol, or a combination thereof.

The pH of the fermentation medium or the fraction thereof is in the range from 2 to 7.

At the end of the demulsification step (a), a stream having an organic phase containing the isoprenoid compound and a phase heavier than the organic phase is obtained, and optionally a solid phase containing host cell debris and cells.

This stream may contain more particularly: an oil organic phase also referred therein as “organic phase” containing the isoprenoid compound, eventually a residual emulsion phase, a heavy phase also referred therein as the “phase heavier than the organic phase” containing mainly water, and eventually, at the bottom, deposits (debris if any and dead cells).

### Organic Compounds

The organics compound envisaged herein can an isoprenoid compound, in particular a terpene or polyterpene, for example farnesene.

Terpenes are a large class of hydrocarbons that are produced in many organisms. They are derived by linking units of isoprene (C₅H₈) and are classified by the number of isoprene units present. Hemiterpenes consist of a single isoprene unit. Isoprene itself is considered the only hemiterpene. Monoterpenes are made of two isoprene units and have the molecular formula C₁₀H₁₆. Examples of monoterpenes are myrcene, geraniol, limonene, and terpineol. Sesquiterpenes are composed of three isoprene units and have the molecular formula C₁₅H₂₄. Examples of sesquiterpenes are farnesene, farnesol, amorpha-4,11-diene and patchoulol. Diterpenes are made of four isoprene units and have the molecular formula C₂₀H₃₂. Examples of diterpenes are cafestol, kahweol, cembrene, and taxadiene. Sesterterpenes are made of five isoprene units and have the molecular formula C₂₅H₄₀. An example of a sesterterpene is geranylfarnesol. Triterpenes consist of six isoprene units and have the molecular formula C₃₀H₄₈. Tetraterpenes contain eight isoprene units and have the molecular formula C₄₀H₆₄. Biologically important tetraterpenes include the acyclic lycopene, the monocyclic gamma-carotene, and the bicyclic alpha- and beta-carotenes. Polyterpenes consist of long chains of many isoprene units. Natural rubber consists of polyisoprene in which the double bonds are cis.

When terpenes are chemically modified (e.g., via oxidation or rearrangement of the carbon skeleton) the resulting compounds are generally referred to as terpenoids, which are also known as isoprenoids.

An isoprenoid compound is typically composed of repeating five-carbon isopentenyl diphosphate (IPP) units, although irregular isoprenoids and polyterpenes have been reported.

The isoprenoid compound produced by the methods described herein can be:
(a) selected from the group consisting of a hemiterpene, monoterpene, sesquiterpene, diterpene, triterpene, tetraterpene, and polyterpene, or
(b) an isoprenoid which is not a carotenoid, or
(c) a C₅-C₂₀ isoprenoid, or
(d) selected from the group consisting of abietadiene, amorphadiene, carene, α-farnesene, β-farnesene, farnesol, geraniol, geranylgeraniol, isoprene, linalool, limonene, myrcene, nerolidol, ocimene, patchoulol, β-pinene, sabinene, γ-terpinene, terpinolene and valencene.

The isoprenoid compound can be a C5-C₂₀ isoprenoid, in particular which may not be a carotenoid.

The isoprenoid compound can be a hemiterpene, e.g., isoprene.

The isoprenoid compound can be a monoterpene and be selected from carene, geraniol, linalool, limonene, myrcene, ocimene, β-pinene, sabinene, γ-terpinene, terpinolene, or a combination thereof.

The isoprenoid compound can be a sesquiterpene and be selected from abietadiene, amorphadiene, farnesene (α-farnesene, β-farnesene), farnesol, nerolidol, patchoulol, valencene, and combinations thereof.

The isoprenoid compound can be a diterpene and can be geranylgeraniol.

The isoprenoid compound can be a sesquiterpene. The isoprenoid compound can be farnesene. The isoprenoid compound can be α-farnesene, β-farnesene or a mixture thereof.

### Overall Recovery Process

The methods described herein comprise the production and recovery of organic compounds, in particular isoprenoid compounds, from a fermentation mixture. A fermentation mixture (also referred to herein as fermenter broth or whole cell broth (WCB)) typically comprises microorganisms, a culture medium and, once cultivation has started, the fermentation products or organic compounds produced by the microorganisms. These organic compounds are preferably released or secreted by the microorganisms in the culture medium as organic compounds, from which they can be recovered.

The methods of recovery described herein comprise the separation of the cellular fraction from the rest of the fermentation medium. This can be done by centrifugation, whereby a microbial pellet is generated and discarded, while the organic phase generally supernatant is used for further extraction of the organic compound. Some of the organic compounds of interest will remain at least in part associated to the microbial host cell during the standard recovery processes (referred to herein as cell-associated organic compounds). These are typically lost in current microbial production processes with the microbial pellet.

The fraction of the fermentation medium from which the cellular fraction has been removed may further be treated for recovery of the organic compound using conventional recovery processes including, but not limited to liquid-liquid separation.

Another source of loss of organic compounds is due to emulsion formation, which is inherent to microbial production systems. Indeed, emulsion formation can be promoted in the fermentation medium by the mechanical energy from fermentation (e.g. from agitators or fermentation gases produced by the microbial host cells), or by the microbial host cells or various bio-molecules therein.

Provided herein are recovery processes for organic compounds from a fermentation mixture characterized in that they comprise the recovery of said organic compounds comprised in emulsions, here emulsions of organic compounds in water.

The methods described herein provide for an improved recovery of organic compounds from a fermentation mixture, which methods comprise recovering the organic compounds which are present in the fermentation medium in emulsions in presence of a salt and a surfactant.

The methods described herein can comprise the steps of providing a fermentation mixture comprising microorganisms, a culture medium and the fermentation products or organic compounds produced by the microorganisms, adding a surfactant to the fermentation vessel to reduce emulsion formation.

### Solid/Liquid Separation Step

The methods described herein can comprise a solid/liquid separation step. The solid/liquid separation step separates the microorganisms from the fermentation mixture. The stream comprising the microorganisms is also referred to herein as “microbial pellet”, or “solid/liquid heavy phase”. This stream comprises the microorganisms and cell-associated organic compounds, and may further comprise host cell debris, culture medium and organic compounds. The solid/liquid heavy phase is mainly containing the biomass. The supernatant or light phase obtained by solid/liquid separation of the fermentation mixture, also referred to herein as “solid/liquid light phase” or “concentrated clarified broth (CCB)”, comprises the culture medium, free organic compounds (organic phase) and organic compounds comprised in an oil, and may further comprise host cell debris. There is also an option to recovery the isoprenoid compound at this solid/liquid separation step from the light phase.

The solid/liquid separation of the fermentation mixture can be achieved by techniques known in the art including, but not limited to, centrifugation, filtration, and decantation, preferably by centrifugation.

A centrifuge can separate the fermentation mixture in batch or on a continuous flow basis. Preferably continuous flow centrifugation is used in the methods described herein. A non-limiting example of a centrifuge suitable for solid/liquid separation of a fermentation mixture as taught herein is a disk stack centrifuge, such as a disk stack centrifuge with nozzles. Centrifugation conditions can be suitably determined by the skilled person to achieve the desired solid/liquid separation.

### Separation Step

This step allows separation of the organic phase obtained in step (a). The organic phase containing the isoprenoid compound is separated by a liquid-liquid separation, such as centrifugation. The liquid/liquid separation step separates the organic compound from the phase heavier than the organic phase. The light phase obtained comprising the organic compound is also referred to herein as “crude”, “organic phase” or “liquid/liquid light phase”. This stream comprises the organic compounds produced by the fermentation and may further comprise some cells, generally dead cells. The heavy phase recovered, also referred to herein as “phase heavier than the organic phase” or “liquid/liquid heavy phase”, comprises the culture medium, cells and may further comprise host-cell debris, free organic compounds and organic compounds comprised in an emulsion. A further solid phase can be obtained, also referred to herein as “discharged phase”, “discharge composition” which comprises the culture medium, host-cell debris, cells and may further comprise free organic compounds and organic compounds comprised in an emulsion.

The liquid/liquid separation can be achieved by known techniques, including, without limitation, centrifugation, filtration, and decantation, preferably centrifugation.

A centrifuge can separate liquid/liquid light phase in batch or on a continuous flow basis. Preferably continuous flow centrifugation is used in the methods described herein. A non-limiting example of a centrifuge suitable for liquid/liquid separation of a fermentation mixture as taught herein is a disk stack centrifuge. Centrifugation conditions can be suitably determined by the skilled person to achieve the desired liquid/liquid separation.

### Extraction of Isoprenoid Compounds Associated in Cells

The phase heavier than the organic phase or the solid phase containing host cell debris and cells obtained in step (b), the solid/liquid heavy phase or the liquid/liquid heavy phase previously mentioned all contain cells (dead or living cells depending on the fraction) and/or host cell debris, which all contains some isoprenoid compounds.

The isoprenoids contained in cells and/or host cell debris of one or several of these phases are extracted and a liquid stream containing the isoprenoid compounds is obtained. This extraction can be obtained by any appropriate techniques known in the art to extract/release an organic compound from a cell and/or host cell debris. These techniques include, but are not limited to, mechanically treating, chemically treating, enzymatically treating, physically treating, or combinations thereof. Mechanically treatment includes, but is not limited to, homogenizing a cell, applying ultrasound to a cell, cold-pressing a cell, milling a cell or the like, and combinations thereof. In a preferred embodiment, milling is used to extract the organic compound from the cells and/or host cell debris.

Mechanically treating a cell can include, but is not limited to, processes utilizing a French pressure cell press, a sonicator, a homogenizer, a ball mill, a rod mill, a pebble mill, a bead mill, a high-pressure grinding roll, a vertical shaft impactor, an industrial blender, a high shear mixer, a paddle mixer, a polytron homogenizer or the like, and combinations thereof.

Physically treatment can include, but is not limited to, heating a cell, decompression, osmotic shock. Heating a cell can include, but is not limited to, resistive heating, convection heating, steam heating, heating in a fluid bath, heating with solar energy, heating with focused solar energy, and the like, any of which can be performed in a tank, pool, tube, conduit, flask, or other containment device.

Chemically treatment includes but is not limited to contacting a cell with a chemical, such as antibiotics, chelating agents, solvents, detergents, chaotropes, or a combination thereof.

Enzymatic lysing refers to lysis of a cell wall or cell membrane of a cell by contacting the cell with one or more enzymes. Enzymatic methods include lytic enzymes or autolysis.

### EXAMPLES

### Example 1

### L-62 Addition in a 2×10 L Fermenter

### Main Procedures

One 10 L fermenter was used as a control (NB5-F-164), and a second 10 L fermenter was used for L-62 addition at 24 hour intervals by adding specific amounts of L-62 just after each withdrawal (NB7-F-164); and a third 10 L fermenter was used for continuous L-62 addition (NB8-F-164) during fermentation. However, due to the pump stroke size, a continuous addition was not suitable, and the addition of L-62 was changed to semi-continuous (addition started by adding specific amount in 1 hour intervals and then changed to 2 hour intervals for convenience). The added L-62 was diluted to 25% by water and filter sterilized. The dilution was necessary for ease of pumping and dosing small amount. In order to make sure no issue originated from inoculation of the cells to 10 L fermenters, no L-62 was added until LH 24 hours. L-62 was added at LH 24 starting with equivalent amount required for the current process, based on withdrawal amount. The amount added decreased between each withdrawal until no addition of L-62 was done at LH 240. L-62 was added again on the next withdrawal for NB7 and at regular 2-hour intervals for NB8 until end of fermentation or harvest.

The aeration in each three fermenters was kept similar at 3 L/min air sparge to the fermenters. Once L-62 addition was started, the biomass and cells oxygen demand increased significantly for NB7 and NB8, and % dissolved oxygen (DO) was on lower scale compared to the control. Percent DO started decreasing and reached zero around LH 96 for NB7 and NB8. Air sparge increased to 6 L/min for NB7 and NB8. No L-62 was added between the 8th and 9th withdrawals. After LH 120, the air feed was increased from 6 L/min to 10 L/min (max for the 10 L fermenter) for NB7 and NB8 while it remained at 3 L/min for the control. This led to increased biomass in the NB7 and NB8 fermenters and at around LH 216, the % dissolved oxygen in NB7 and NB8 started falling to zero again. At LH 288, NB7 and NB8 were supplemented with 2-3 L/min oxygen to boost dissolved oxygen level.

The effect of volumetric biomass and emulsion or crude oil were analyzed by capillary centrifugation of samples from each withdrawal and harvest.

### Effect of L-62 Addition on Biomass and Oxygen Demand

The cell viability and biomass amount were similar at LH24 (before L-62 addition) for all three fermenters. Once L-62 addition was started, biomass and oxygen demand increased for NB7 and NB8. Once L-62 was added, samples from the next withdrawals showed oil in the whole cell broth and direct centrifugation was enough to recover the crude oil (e.g., no need for DSP demulsification). Increasing aeration from 3 L/min to 10 L/min was not enough to keep DO above zero % as the rate of cell growth increased significantly after L-62 was added. Supplementing with continuous ~3 L/min pure oxygen helped to keep dissolved oxygen above 15%.

### Effect of L-62 Addition on Crude Oil

NB7 and NB8 had oil in the samples after L-62 addition, while the control had emulsion as expected. This demonstrated that it was possible to block emulsion formation or complete demulsification in the fermenter, during fermentation.

From LH 24 to LH 240, L-62 addition decreased each day, and no addition was done between 8^{th} and 9^{th} withdrawal. Sample from 9th withdrawal showed no oil (all emulsion). The L-62 addition started again and on the next (10^{th}) withdrawal, and oil was observed again. This confirms the effect of L-62 addition on obtaining oil from the whole cell broth.

### Effect of L-62 Addition on Withdrawal Volume

The withdrawal from each NB7 and NB8 increased proportional to biomass and % dissolved oxygen which is also affected by L-62 addition. The withdrawal volume increased from LH24 to LH 96 and decreased until aeration increased. The maximum withdrawal volume obtained after aeration feed increased to maximum 10 L/min. The withdrawal started decreasing until pure oxygen supplementation, a day from harvest. The minimum amount of L-62 needed to block emulsion were used as a base line for minimum requirement and helped in estimating L-62 consumption reduction.

### Effect of L-62 Addition on Total Volume of Crude Oil

As the withdrawal volume increases, so does the total volume of crude oil withdrawal. For the most part, the graph of total crude oil is similar to the graph of total biomass. No crude oil was observed in the whole cell broth when L-62 addition was stopped at LH 240.

### Effect of L-62 Addition on Titer of Crude Oil

L-62 addition increased the biomass and hence the withdrawal volume increased. This increased total crude oil but slightly decreased the crude oil per withdrawal weight (equivalent to titer). In most of the samples, the titer appeared equivalent to the control.

### Effect of L-62 Addition on Quality of the Crude Oil

The color of the crude oil obtained in the lab appeared lighter compared to the control. GC data showed that crude oil from L-62 addition had better purity than the control.

Effect of L-62 Addition into the Fermenter on Surfactant Consumption

The L-62 addition started around 1.5 g per withdrawal kg and gradually decreased to zero and increased again to up to 0.5 g per withdrawal. Based on the consumption rate observed, the overall consumption of L-62 based on whole cell broth reduced by 83% for NB7 and 82% for NB8. Based on lab demulsification of sample from the control, the overall consumption of L-62 was reduced by 88% for both NB7 and NB8.

### Effect of L-62 Addition on Yield on Sugar.

A re-calculation and data from fermentation showed about 2% yield loss for surfactant added inside fermenter compared to control. The semi-continuous addition of L-62 had a slight edge regarding productivity and recovery compared to the one-time addition. L-62 addition significantly increased the amount of whole cell broth (WCB). The rate of crude oil recovered increased by 24% for NB7 and 32% for NB8.

### Example 2

### L-62 Addition to 10 L Fermenter

### Main Procedure

The aeration feed for the control fermenter remained the same as EXAMPLE 1 at 3 L/min. The aeration for fermenters NB7 (a onetime L62 addition between withdrawals) and NB8 (30% L62 needed added at once and the remaining at semi-continuous addition mode) was set at 10 L/min and supplemented with pure oxygen starting from the beginning of the run. L-62 addition started after LH24. L-62 addition was kept constant throughout the 12-day run by adding about 30% of the requirement at one time and followed by addition of the remaining every 2 hours until next withdrawal. NB7 was kept same condition as NB8.

### Effect of L-62 Addition on Biomass.

There was a slight difference on biomass amount at LH24 between NB8 and control (before L-62 addition). Once L-62 addition was started, biomass increased significantly for NB8. Based on measurement by the capillary tube, the biomass for NB7, where aeration was similar to NB8 (at 10 L/min plus pure oxygen supplementation), was slightly lower than NB8. The dissolved oxygen level remained higher in NB7 at around 50% while dissolved oxygen for NB8 was around 25% for most of the run.

### Effect of L-62 Addition on Crude Oil.

For L-62 added fermenter, crude oil was obtained in the whole cell broth throughout the run. The inventor observed the crude oil obtained increasing v/v in the sample obtained from withdrawals until the fifth day (LH 96) and became constant v/v after that until harvest.

### Effect of L-62 Addition on Withdrawal Amount.

L-62 addition increased the amount of withdrawal significantly (NB8). Aeration with pure oxygen supplement also produced equivalent amount of withdrawal (NB7).

### Example 3

### Effect of L-62 Addition on Dsp Recovery

### Main Procedure: (Equivalent of Adding L-62 to Harvest Tank)

From fermenter NB5 and NB6, about 420 grams was sampled from the 5th withdrawal and heated to 75° C., and 0.4% w/w L-62 added for demulsification. The samples were mixed for 2 hours and centrifuged. The four phases (oil, Dead Cell Layer (DCL), Aqueous layer and S/L HP layer) were decanted, collected, and weighed. Samples from each phase including starting WCB were analyzed by GC. From fermenter NB8 5^{th} withdrawal, sample was centrifuged as is, and the four phases were similarly separated. The phases were analyzed by Gas Chromatography (GC) for farnesene oil (“fene oil”) distribution in each phases.

### Results and Observations

The overall recovery for NB5 (Control) and NB6 was lower than current recovery (~85% recovery and the overall loss, ~15%. current recovery ~88%). The loss distribution for control was lower in the L/L HP and higher in S/L HP, while the current process has the opposite. L-62 addition has relatively lower losses in both S/L HP and L/L HP. Compared to L-62 addition to harvest tank, the overall losses from the L-62 addition (NB8) are reduced by almost 45-50%. As compared to current process, the overall losses for L-62 addition reduced by ~25%. The overall recovery for L-62 addition to harvest tank was around 82-85%, while adding L-62 inside fermenter had ~88-91% recovery. In comparison, current recovery is around 88%.

TABLE 1
DSP recovery comparing adding surfactant inside fermenter
versus adding surfactant into harvested fermentation broth

| | | | | | | | | | |
|---|---|---|---|---|---|---|---|---|---|
| | | | | | | Sum | Fene | | |
| | | | Material | | | fene oil | mass | | |
| | | Sum of | mass | | Net | in 4 | balance | % | % |
| Sample | Start | phases- | balance | Titer | fene | phases- | deviation- | recovery | total |
| name | wt-g | g | deviation | g/kg | oil-g | g | % | or loss | losses |

NB5-F-165-5^{th} Withdrawal- demulsification mechanism
equivalent to adding L62 to harvest tank

| | | | | | | | | | |
|---|---|---|---|---|---|---|---|---|---|
| WCB | 395.26 | 382.50 | −3.23 | 144.68 | 57.18 | 54.27 | −5.09 | | 14.2 |
| Crude Oil | 47.46 | | | 971.96 | 46.13 | | | 80.7 | |
| DCL | 25.35 | | | 71.05 | 1.80 | | | 3.1 | |
| Aqueous | 257.52 | | | 1.06 | 0.27 | | | 0.5 | |
| Phase | | | | | | | | | |
| S/L HP | 82.17 | | | 73.90 | 6.07 | | | 10.6 | |

NB6-F-165-5^{th} Withdrawal- demulsification mechanism
equivalent to adding L62 to harvest tank

| | | | | | | | | | |
|---|---|---|---|---|---|---|---|---|---|
| WCB | 402.4 | 393.04 | −2.33 | 151.94 | 61.14 | 59.54 | −2.62 | | 14.6 |
| Crude Oil | 51.35 | | | 985.16 | 50.59 | | | 82.7 | |
| DCL | 16.33 | | | 82.36 | 1.35 | | | 2.2 | |
| Aqueous | 251.79 | | | 1.12 | 0.28 | | | 0.5 | |
| Phase | | | | | | | | | |
| S/L HP | 98.57 | | | 74.31 | 7.32 | | | 12.0 | |

NB8-F-165-5^{th} Withdrawal- demulsification mechanism
equivalent to adding L62 to harvest tank

| | | | | | | | | | |
|---|---|---|---|---|---|---|---|---|---|
| WCB | 498.81 | 487.59 | −2.25 | 138.12 | 68.89 | 72.34 | 5.00 | | 6.9 |
| Crude Oil | 67.79 | | | 996.73 | 67.57 | | | 98.1 | |
| DCL | 18.54 | | | 111.92 | 2.07 | | | 3.0 | |
| Aqueous | 233.21 | | | 1.07 | 0.25 | | | 0.4 | |
| Phase | | | | | | | | | |
| S/L HP | 213.05 | | | 11.49 | 2.45 | | | 3.6 | |

### Example 4

### Effect of L-62 Addition on Downstream Process (Dsp) Recovery

### Procedures

For NB5, withdrawals and harvest from batch 165 mixed in tank. About 420 μm sampled and demulsified at 75° C. in the lab, with addition of 0.8% L-62 and centrifuged. For NB8, sample from batch 165 was centrifuged in the lab. The four phases were separated, weighed and analyzed on GC.

### Results

For NB5, the recovery of 85.9% was lower than current process but the overall losses of 17.2% was also higher. For NB5, the loss distribution was equivalent to current processes where the L/L HP losses in current processes are around 8-10%, while here it was around 8.7%. For NB5, the amount of L-62 needed was double that of current consumption rate. For NB8, the overall recovery based on crude oil was on the higher end while the overall losses were close to overall losses obtained from withdrawal. For NB8, the loss distribution for DCL is much lower than losses in NB5 or current process.

TABLE 2
DSP recovery and losses for surfactant added inside fermenter during
fermentation versus surfactant added in downstream process

| NB5-1770-Withdrawal and Harvest WCB were mixed at MiniPilot | | | | | | | | | |
|---|---|---|---|---|---|---|---|---|---|
| and sample demulsified and centrifuged in lab | | | | | | | | | |
| Sample | | | Material | | | Sum | Fene | | |
| name | | | mass | | | fene oil | mass | | |
| NB5-F- | | Sum of | balance | | Net | in 4 | balance | % | |
| 1770- | Start | phases- | deviation- | Titer | fene | phases- | deviation- | recovery | % total |
| Mix | wt-g | g | % | g/kg | oil-g | g | % | or loss | Losses |
| Starting | 415.80 | 415.28 | −0.13 | 129.20 | 53.72 | 55.35 | 3.03 | | 17.2 |
| WCB | | | | | | | | | |
| Crude Oil | 47.43 | | | 972.44 | 46.12 | | | 85.9 | |
| DCL | 74.16 | | | 63.00 | 4.67 | | | 8.7 | |
| Aqueous | 250.54 | | | 3.40 | 0.85 | | | 1.6 | |
| phase | | | | | | | | | |
| S/L HP | 88.15 | | | 42.03 | 3.71 | | | 6.9 | |
| NB8-1770-Withdrawal and Harvest WCB were mixed at MiniPilot | | | | | | | | | |
| and sample demulsified and centrifuged in lab | | | | | | | | | |
| Sample | | | Material | | | Sum | Fene | | |
| name | | | mass | | | fene oil | mass | | |
| NB8-F- | | Sum of | balance | | Net | in 4 | balance | % | |
| 1770- | Start | phases- | deviation- | Titer | fene | phases- | deviation- | recovery | % total |
| Mix | wt-g | g | % | g/kg | oil-g | g | % | or loss | Losses |
| Starting | 405 | 404.95 | −0.01 | 129.44 | 52.42 | 54.37 | 3.72 | | 9.3 |
| WCB | | | | | | | | | |
| Crude Oil | 50.53 | | | 979.33 | 49.49 | | | 94.4 | |
| DCL | 19.33 | | | 69.93 | 1.35 | | | 2.6 | |
| Aqueous | 217.60 | | | 1.80 | 0.39 | | | 0.7 | |
| phase | | | | | | | | | |
| S/L HP | 152.49 | | | 20.63 | 3.15 | | | 6.0 | |

### Example 5

### Effect of “Heating “Harvested Fermentation Broth from L-62 Added Inside Fermenter on Dsp Recovery

### Main Procedures

For NB5, withdrawals and harvest from batch 165 mixed in tank at mini pilot. About 400 μm WCB sampled and demulsified in the lab at 75° C. with 0.8% L-62 addition and centrifuged. For NB8, WCB sample from batch 165 was heated to 75° C. and centrifuged in the lab. The two phases from each sample were separated, weighed and analyzed on GC based on purity of the crude oil.

### Result and Observation

For NB5, the overall recovery is similar to other lab scale runs and lower than current process. For NB8, the overall recovery was 88% and equivalent to current process. The crude oil from L-62 addition showed more purity than the control.

TABLE 3
Effect of heating fermentation broth from surfactant added inside fermenter on DSP recovery

| | Start | WT - | | Starting | | Purity | Net | |
|---|---|---|---|---|---|---|---|---|
| | wt - | HP - | Titer - | net crude | Crude oil | % - | Crude | % |
| Samples name | G | G | g/kg | oil-g | recovered-g | w/w | oil-g | Recovery |
| AEXM-NB5-F- | 395.9 | 340.6 | 129.2 | 51.16 | 44.31 | 94.62 | 41.9 | 82.0 |
| 1770 #2- | | | | | | | | |
| Demulsified in | | | | | | | | |
| lab- 75 C. | | | | | | | | |
| AEXM-NB8-F- | 377.0 | 327.6 | 129.4 | 48.78 | 44.10 | 97.50 | 43.0 | 88.1 |
| 1770 #3-heated | | | | | | | | |
| to 75 C. and | | | | | | | | |
| centrifuged in lab | | | | | | | | |

### Example 6

### Effect of Heat Sterilization of Surfactant on its Potency

### Main Procedure

WCB obtained from 10 L fermenter (NB5-F-1774) used as a control for Strain development. This fermenter run was extended for two days to test autoclaved L-62.

### Results

When WCB centrifuged as is, the Loss in S/L HP was equivalent to current process. When WCB demulsified equivalent to adding L-62 to harvest Tank, the loss in S/L HP is much higher than current process. When CCB was separated and demulsified similar to current process, the overall losses were equivalent to current process: Table 5. L-62 added WCB had about 2.5% lower losses compared to current process: Table 5.

TABLE 4
Heat sterilized surfactant demulsification at DSP with condition similar to
current process and with a scenario of adding surfactant to harvest tank.

| | | | | | | | | | |
|---|---|---|---|---|---|---|---|---|---|
| | | | Material | | | Sum | Fene | | |
| | | | mass | | Net | fene oil | mass | % | % |
| Sample | Start | Sum of | balance | Titer | fene | in 3 | balance | recovery | total |
| name | wt-g | phases | deviation | g/kg | oil | phases | deviation | or loss | losses |

NB5-F-174 #1: End of strain test that was used as a control centrifuged as is and the S/L HP and CCB
separated. Analyzed same day as it was sampled for fene oil distribution

| NB5-F- | 94.10 | 93.82 | −0.30 | 148.81 | 14.00 | 13.84 | −1.19 | | |
|---|---|---|---|---|---|---|---|---|---|
| 174-WCB | | | | | | | | | |
| NB5-F-174 | 42.78 | | | 314.37 | 13.45 | | | 96.0 | |
| #1-CCB | | | | | | | | | |
| NB5-F-174 | 51.04 | | | 7.59 | 0.39 | | | 2.8 | |
| #1-S/L HP | | | | | | | | | |
| NB5-F-174 #2: End of strain test was used as a control sampled and, | | | | | | | | | |
| demulsified in the lab. The three phases separated and analysed for Fene Oil | | | | | | | | | |
| distribution (demulsification similar to adding L62 to Harvest Tank) | | | | | | | | | |
| NB5-F- | 414.43 | 408.04 | −1.54 | 148.81 | 61.67 | 59.07 | −4.22 | | |
| 174-WCB | | | | | | | | | |
| NB5-F-174- | 50.76 | | | 973.81 | 49.43 | | | 80.2 | |
| #2-Crude | | | | | | | | | |
| Oil | | | | | | | | | |
| NB5-F- | 119.78 | | | 43.70 | 5.23 | | | 8.5 | |
| 174- #2- | | | | | | | | | |
| DCL | | | | | | | | | |
| NB5-F- | 237.50 | | | 18.56 | 4.41 | | | 7.1 | 15.6 |
| 174-#2-S/L | | | | | | | | | |
| HP | | | | | | | | | |

TABLE 5
Heat sterilized surfactant demulsification at DSP with condition similar
to current process and with adding surfactant inside fermenter

| | | | | | | | | | |
|---|---|---|---|---|---|---|---|---|---|
| | | | Material | | | Sum | Fene | | |
| | | | mass | | Net | fene oil | mass | % | % |
| Sample | Start | Sum of | balance | Titer | fene | in 3 | balance | recovery | total |
| name | wt-g | phases | deviation | g/kg | oil | phases | deviation | or loss | losses |

NB5-F-174 #3: End of strain test that was used as a control centrifuged as is and the S/L HP and CCB
separated. CCB was demulsified. Crude oil, DCL and HP separated and analyzed for fene oil distribution.

| NB5-F-174 | 654.49 | 654.49 | 0.00 | 148.88 | 97.44 | 88.73 | −8.95 | | |
|---|---|---|---|---|---|---|---|---|---|
| #3-WCB | | | | | | | | | |
| NB5-F-174 | 79.58 | | | 968.93 | 77.11 | | | 79.1 | |
| #3-Crude | | | | | | | | | |
| Oil | | | | | | | | | |
| NB5-F- | 257.00 | | | 32.12 | 8.26 | | | 8.5 | |
| 174#3-DCL | | | | | | | | | |
| NB5-F-174 | 317.91 | | | 10.58 | 3.36 | | | 3.5 | 11.9 |
| #3-S/L HP | | | | | | | | | |
| NB5-F-174 #2: End of strain test that was used as a control sampled and | | | | | | | | | |
| demulsified in the lab. The three phases separated and analysed for Fene Oil | | | | | | | | | |
| distribution (demulsification similar to adding L62 to Harvest Tank) | | | | | | | | | |
| NB5-F-174 | 191.97 | 192.10 | 0.07 | 154.55 | 29.67 | 29.38 | −0.98 | | |
| # 4-WCB | | | | | | | | | |
| NB5-F-174- | 26.78 | | | 990.92 | 26.54 | | | 89.4 | |
| #4-Crude | | | | | | | | | |
| Oil | | | | | | | | | |
| NB5-F- | 48.82 | | | 37.92 | 1.85 | | | 6.2 | |
| 174- #4- | | | | | | | | | |
| DCL | | | | | | | | | |
| NB5-F- | 116.50 | | | 8.50 | 0.99 | | | 3.3 | 9.6 |
| 174-#4-S/L | | | | | | | | | |
| HP | | | | | | | | | |

### Example 7

### Effect of Steam Sterilization (Autoclave)

### On L-62 Demulsification Efficacy

### Main Procedure

L-62 that was diluted to 25% by water and filter sterilized (used for the 1st batches of 10 L fermentation run) was autoclaved. On cooling, two phase separation observed. The phases were mixed and sample aliquoted for bench scale demulsification trial on 450 μm sample heated to 75° C. (Table 4). The remaining autoclaved L-62 was tested by adding in the 10 L fermenter. A strain development work was run and at end of the experiment, the control (NB5-F-1774) was used to test efficacy of autoclaved L-62 for two days. Table 5.

### Results and Observations

Diluted to 25% by water and Autoclaved L-62 showed phase separation upon cooling. After mixing no phase separation observed for 24 hours. After few days, phase separation observed again. Equivalent amount of autoclaved L-62 needed as regular L-62 to demulsify on bench scale. Equivalent amount of autoclaved L-62 needed as filter sterilized L-62 to demulsify in the fermenter.

### CONCLUSIONS

The inventors found that by adding L-62 in the fermenter effectively blocked emulsion formation and/or completed demulsification in the fermenter.

Adding L-62 in the fermenter reduced surfactant consumption by 91%. This represents a significant cost savings versus current processes in use in the industry.

The L-62 addition in the fermenter will reduce two steps centrifugation to single step centrifugations. The L/L centrifuge can be used as a purification step, based on the crude oil characteristics from S/L centrifuge.

Adding L-62 into the fermenter eliminates the need for heating CCB to 75-80° C.

Adding L-62 in the 10 L fermenter increased overall recovery by 2-4% at DSP. This represents a significant cost savings versus current processes in use in the industry.

Adding L-62 in the fermenter increased productivity at DSP significantly (up to 70% on the optimized run with max air flow and oxygen supplementation: keeping DO~30%).

At a rate of 10 L optimized run (DO>30%), adding L-62 in the fermenter may slightly decrease farnesene oil yield on sugar.

### Example 8

### 10 L Run

Two 10 L fermenters prepared to test surfactant addition inside fermenter with conditions equivalent to current production. NB2-F-177 was used as a control and NB5-F-177 was used as the experimental fermenter. Air flow was set at 3 L/min and L-62 addition started at LH24. About 30% of the estimated required L-62 added at LH24 and the remaining 70% added in equal amount every 2 hours until 2 hours before next harvest. These procedures were repeated after each withdrawal until the final harvest.

Each withdrawal and the final harvest collected and processed. Whole cell broth from control was centrifuged and CCB collected. Sample from CCB was taken to lab and demulsified by heating to 75° C. and adding L-62 equivalent to current production process. WCB from L-62 addition was centrifuged and oil with high moisture collected. Sample from this oil was recentrifuged in the lab and crude oil recovered.

Data from lab and pilot process showed a farnesene oil 90% recovery and 7.9% loss in DCL for control and 88% recovery for the L-62 addition inside the fermenter. Data from lab demulsification of WCB showed 79% recovery, 6% loss in DCL and 7.8% loss in S/L HP. As demonstrated in previous experiments, adding L-62 to WCB (equivalent of adding L-62 to harvest tank) showed less recovery of product. On the other hand centrifugation of WCB in the lab from L-62 addition inside fermenter showed 88% recovery of product, 3.5% loss in DCL and 6.3% loss in S/L HP.

All references cited in this specification are herein incorporated by reference as though each reference was specifically and individually indicated to be incorporated by reference. The citation of any reference is for its disclosure prior to the filing date and should not be construed as an admission that the present disclosure is not entitled to antedate such reference by virtue of prior invention.

It will be understood that each of the elements described above, or two or more together may also find a useful application in other types of methods differing from the type described above. Without further analysis, the foregoing will so fully reveal the gist of the present disclosure that others can, by applying current knowledge, readily adapt it for various applications without omitting features that, from the standpoint of prior art, fairly constitute essential characteristics of the generic or specific aspects of this disclosure set forth in the appended claims. The foregoing embodiments are presented by way of example only; the scope of the present disclosure is to be limited only by the following claims.

1. A method for reducing emulsion formation inside a fermenter during the production of organic compounds comprising
(a) fermenting a microorganism with a feed stock in a fermentation vessel to produce a whole cell broth (WCB) comprising organic compounds;
(b) adding a surfactant to the feed stock, whole cell broth, to a portion of the whole cell broth, or a combination thereof, in the fermentation vessel; and
(c) optionally, removing a portion of the whole cell broth;
(d) heating the fermentation vessel.

2. The method of claim 1, wherein the microorganism is a bacteria, fungi, or algae, wherein:
the bacteria preferably is E. coli, a Bacillus or Lactobacillus species;
the fungi preferably is a yeast such as a Saccharomyces or Pichia species; and
the algae is preferably a chlorella species.

3. The method of claim 1, wherein the microorganism is a recombinant microorganism.

4. The method of claim 1, wherein the surfactant is selected from an anionic surfactant, a non-ionic surfactant, or a combination thereof;
wherein the an anionic surfactant is selected from one or more of sulfates and sulfonates, sodium dodecylsulfate (SDS), sodium dodecylbenzene sulfonate, a disulfonate, sodium dodecylnaphthalene sulfate, dialkyl benzenealkyl sulfates and sulfonates, acids optionally abitic acid, alkyldiphenyloxide disulfonate including two C6-C16 hydrocarbonchains branched on each of the sulfonated aromatic rings, and combinations thereof; and
the non-ionic surfactant is selected from polyether polyols, optionally copolymers of propylene oxide (PO) and ethylene oxide (EO), including those made by capping polypropylene glycols with ethylene oxide.

5. The method of claim 4, wherein the non-ionic surfactant is a polyether glycol having a molecular weight of at least 2400 g/mol, less than 2800 g/mol, or less than 3000 g/mol; and/or the anionic surfactant is selected from sulfates and sulfonates.

6. The method of claim 1, wherein the surfactant is L-62 (polyether polyol) or L-81 (polyether polyol).

7. The method of claim 1, wherein the surfactant is added in an amount of between about 0.001% v/v (volume/volume) and 1% v/v (volume/volume), more preferably between about 0.01 and 1% v/v, more preferably between about 0.02 and 0.06% v/v, or between 0.03 and 0.05% v/v.

8. The method of claim 1, wherein the fermentation lasts for between about 1 and 14 days, and wherein a portion of the whole cell broth is removed after 1 day.

9. The method of claim 1, wherein step (b) lasts for about 24 hours, and wherein the method further comprises isolating organic compounds from the whole sell broth.

10. The method of claim 1, wherein the organic compounds comprise isoprenoids, wherein at the isoprenoids are:
(a) selected from the group consisting of a hemiterpene, monoterpene, sesquiterpene, diterpene, triterpene, tetraterpene, and polyterpene;
(b) an isoprenoid which is not a carotenoid;
(c) a C₅-C₂₀ isoprenoid; or
(d) selected from the group consisting of abietadiene, amorphadiene, carene, α-farnesene, β-farnesene, farnesol, geraniol, geranylgeraniol, isoprene, linalool, limonene, myrcene, nerolidol, ocimene, patchoulol, β-pinene, sabinene, γ-terpinene, terpinolene and valencene.

11. The method of claim 1, wherein the organic compounds comprise, consist of, or consist essentially of farnesene.

12. The method of claim 1, wherein the method further comprises
separating the whole cell broth into an organic phase comprising the isoprenoid compound and a heavy phase comprising solid and liquid fermentation medium; and
purification of the isoprenoid compound.

13. The method of claim 1, wherein the method comprises an air feed to fermenter between about from 3 L/minutes to 10 L/minutes, and/or wherein the dissolved oxygen in the fermenter is maintained at above about 15%.

14. The method of claim 1, wherein the dissolved oxygen in the fermenter is maintained at 0%.

15. The method of claim 1, wherein the surfactant reduces emulsion formation between about 10%, 20%, 30%, 40%, 50%, 60%, 80%, or 90% as compared to emulsion formation in a fermenter without the addition of the surfactant.
